(19) United States
(12) Patent Application Publication
(10) Pub. No.: US 2022/0118168 A1
(43) Pub. Date: Apr. 21, 2022

(54) LAVAGE SYSTEMS AND DEVICES HAVING MULTIFUNCTIONAL APPLICATION MEMBER

(71) Applicant: CareFusion 2200, Inc., San Diego, CA (US)

(72) Inventors: Christopher McGinley, Highland Park, IL (US); John Krueger, Muskego, WI (US); Maleeha Mashiatulla, Glen Ellyn, IL (US); Dan Zimbler, Chicago, IL (US)

(21) Appl. No.: 17/501,752

(22) Filed: Oct. 14, 2021

Related U.S. Application Data
(60) Provisional application No. 63/092,248, filed on Oct. 15, 2020.

(51) Int. Cl. A61M 3/02; B08B 3/04

(52) U.S. Cl. CPC A61M 3/0245; A61M 2205/587; A61M 3/0279; B08B 3/04

(57) ABSTRACT

A system for applying a lavage fluid to a surface, the system having a body configured to house a lavage fluid and a multifunctional application member in fluid communication with the body, wherein the multifunctional application member is configured to dispense the lavage fluid, and wherein the multifunctional application member is configured to provide more than one selectable function.

### CROSS-REFERENCE TO RELATED APPLICATION

This application claims priority to U.S. Provisional Application No. 63/092,248, filed Oct. 15, 2020, the disclosure of which is expressly incorporated herein by reference in its entirety.

### TECHNICAL FIELD

The present disclosure is directed to devices and systems for applying a lavage fluid to a surface, and in particular, devices and systems comprising at least one multifunctional application member.

### BACKGROUND

Currently, lavage (that is, irrigating a body cavity, surgical cavity, or external wound with a medically acceptable fluid) is often employed to prevent contamination of an open surgical wound, which may occur for a variety of reasons such as accidental visceral entry or perforated viscus, operations complicated by gross spillage, departure from sterile technique, and/or existing, ongoing clinical infection. Lavage processes are thus often employed to provide intraoperative antiseptic wound irrigation.

The art of lavage currently embraces a wide variety of different approaches that vary based on the situation (for example, the size and shape of the cavity or wound) and on the medical practitioner performing the lavage process (for example, a practitioner's technique preference). Currently, no specific lavage technique is standard in the art, and as such, medical facilities often require numerous different lavage devices and systems to accommodate the variety of potential approaches. The presentation of such devices and systems is also sometimes a concern, as the inadvertently inappropriate use of such devices and systems (e.g., intravenously, if the device and/or system has a similar appearance to an intravenous device and/or system) could results in devastating effects.

Moreover, several drawbacks exist with current lavage practices, including insufficiencies in antiseptic fluid properties (e.g., the amount of time necessary for an antiseptic fluid to achieve an acceptable biological effect, which may be prohibitive), the risk of systemic absorption of the antiseptic fluid, adverse reactions such as anaphylaxis, peritoneal adhesions, neurotoxicity, and respiratory insufficiency, and improper dosage or contamination of the antiseptic fluid, which are sometimes prepared ad hoc by a medical practitioner performing the lavage.

In addition, the effectiveness of lavage processes, particularly lavage processes performed before and/or during a surgical procedure, is often dependent on the complete or near complete removal of debris and/or microorganisms present within a surgical cavity, and/or the removal of spent lavage fluid. This often poses significant challenges, as surgical sites are frequently irregularly shaped and thus difficult to access and/or see. For example, a surgical site may comprise an area in which tissue is folded or wrinkled. In addition, in the case wherein the surgical site comprises an organ such as the intestine, the surgical site may be resting on other tissue and may not be readily assessable and/or visible without moving the tissue.

To perform current lavage processes in these scenarios, medical practitioners must move and/or remove obstructions (for example, by hand or with a separate instrument) or must forego irrigating the inaccessible areas, often to the detriment of the patient. However, physically moving and/or removing obstructions requires an additional step, which poses time concerns, and is sometimes not possible if the surrounding area is inaccessible to the practitioner's hand or instrument (e.g., when the surrounding area is very small). It is therefore often difficult to remove debris and/or microorganisms efficiently and completely, particularly in areas that are difficult to reach and/or visualize.

There is thus a need in the art for versatile devices and systems for performing lavage processes, and in particular, devices and systems that enable medical practitioners to safely and effectively reduce contamination in surgical wounds that are susceptible to surgical site infections.

### SUMMARY

The present disclosure is directed to devices and systems for delivering a lavage fluid, such as an antiseptic solution, to a surface. The device comprises a body that is configured to house a lavage fluid, such as an antiseptic solution. The body is further configured to be in fluid communication with at least one application member, wherein the at least one application member is configured to apply the lavage fluid to a surface sufficient for a lavage process. According to some aspects, the at least one application member may comprise a multifunctional application member as will be described herein. The devices and systems may be adaptable such that a user may select from two or more different fluid flow rates, fluid flow patterns, and/or fluid flow forces, thus providing selectable control of lavage fluid delivery to a surface. The present disclosure is also directed to methods of using the devices and systems described herein.

### BRIEF DESCRIPTION OF THE DRAWINGS

FIG. 1A shows an example of a compressible body according to aspects of the present disclosure.

FIG. 1B shows an example of a collapsible body according to aspects of the present disclosure.

FIG. 2A shows an example of a body according to aspects of the present disclosure.

FIG. 2B shows an example of a body according to aspects of the present disclosure.

FIG. 3 shows an example of a body's connection portion according to aspects of the present disclosure.

FIG. 4 shows an example of a body with an outer casing according to aspects of the present disclosure.

FIG. 5 shows an example application member according to aspects of the present disclosure.

FIG. 6 shows an example system according to aspects of the present disclosure.

FIG. 7A shows an example dispensing aid according to aspects of the present disclosure.

FIG. 7B shows an example dispensing aid according to aspects of the present disclosure.

FIG. 8 shows an example system according to aspects of the present disclosure.

FIG. 9 shows an example system according to aspects of the present disclosure.

FIG. 10 shows an example system according to aspects of the present disclosure.

FIG. 11 shows an example system according to aspects of the present disclosure.

FIG. 12 shows an example system with more than one nozzle according to aspects of the present disclosure.

FIG. 13A shows an example of a multifunctional application member having a shape configured for acceptable obstruction maneuvering, according to aspects of the present disclosure.

FIG. 13B shows an example of a multifunctional application member having a shape configured for acceptable obstruction maneuvering, according to aspects of the present disclosure.

FIG. 14A shows an example of a multifunctional application member having a shape configured for acceptable obstruction maneuvering, according to aspects of the present disclosure.

FIG. 14B shows an example of a multifunctional application member having a shape configured for acceptable obstruction maneuvering, according to aspects of the present disclosure.

FIG. 14C shows an example of a multifunctional application member having a shape configured for acceptable obstruction maneuvering, according to aspects of the present disclosure.

FIG. 15 shows an example application member comprising a semi-flexible conduit according to aspects of the present disclosure.

FIG. 16A shows an example of an application member comprising an obstruction component according to aspects of the present disclosure.

FIG. 16B shows an example of an application member comprising an obstruction component according to aspects of the present disclosure.

FIG. 17A shows an example of an application member comprising a suction component according to aspects of the present disclosure.

FIG. 17B shows an example of an application member comprising a suction component according to aspects of the present disclosure.

FIG. 18 shows an example of an application member comprising a light component according to aspects of the present disclosure.

FIG. 19 shows an example of an application member comprising a camera component according to aspects of the present disclosure.

### DETAILED DESCRIPTION

The present disclosure is directed to devices and systems for delivering a lavage fluid, such as an antiseptic solution, to a surface. The device comprises a body that is configured to house a lavage fluid, such as an antiseptic solution. The body is further configured to be in fluid communication with at least one application member, wherein the at least one application member is configured to apply the lavage fluid to a surface sufficient for a lavage process. According to some aspects, the at least one application member may comprise a multifunctional application member as will be described herein. The devices and systems may be adaptable such that a user may select from two or more different fluid flow rates, fluid flow patterns, and/or fluid flow forces, thus providing selectable control of lavage fluid delivery. The present disclosure is also directed to methods of using the devices and systems described herein.

As used herein, the term “lavage fluid” refers to a fluid suitable for a lavage process as described herein. As used herein, “lavage” refers to the irrigation of a body cavity, a surgical cavity, and/or an external wound.

According to some aspects, the lavage fluid may comprise an antiseptic solution. As used herein, an “antiseptic solution” refers to a solution comprising at least a solvent and one or more antiseptic agents. According to some aspects, the antiseptic solution is an aqueous solution. As used herein, the term “aqueous solution” refers to a solution wherein the solvent comprises at least a majority of water. It should be understood that in some examples, the solvent may consist of water. According to some aspects, the antiseptic solution is an alcoholic solution. As used herein, the term “alcoholic solution” refers to a solution wherein the solvent comprises at least a majority of alcohol. It should be understood that in some examples, the solvent may consist of one or more alcohols. Non-limiting examples of alcohols include, but are not limited to, ethanol, isopropyl alcohol, n-propanol, and combinations thereof.

In one non-limiting example, the antiseptic agent may comprise a cationic molecule (i.e., a molecule having a positive charge), such as a cationic surfactant or a cationic biguanide derivative (i.e., a compound derived from biguanide). According to some aspects, the antiseptic agent may comprise a bis-(dihydropyridinyl)-decane derivative (i.e., a compound derived from bis-(dihydropyridinyl)-decane). According to some aspects, the antiseptic agent may comprise an octenidine salt and/or a chlorhexidine salt. According to some aspects, the antiseptic agent may comprise alexidine, octenidine dihydrochloride, chlorhexidine gluconate, or a combination thereof.

Additionally or alternatively, the antiseptic agent may comprise iodine. According to some aspects, the iodine may be provided as an iodine complex, such as povidone-iodine (PVPI), nonylphenoxy-(ethyleneoxy)-iodine, polyethylene oxy polyprop leneoxy-iodine, undecoylinium-chloride-iodine, iodine povacrylex, and combinations thereof.

Additionally or alternatively, the antiseptic agent may comprise an oxidant (i.e., an oxidizing agent). Non-limiting examples of oxidants according to the present disclosure include, but are not limited to, sodium hypochlorite, hydrogen peroxide, and combinations thereof.

The antiseptic agent may have an antimicrobial activity sufficient to provide an acceptable log reduction of microbes in a certain time period. It should be understood that as used herein, the term “microbes” may refer to any microorganism to be killed and/or removed as a result of lavage. Example microbes include bacteria, fungi, viruses, and combinations thereof.

Example bacteria include, but are not limited to, Streptococcus mutans, S. pyogenes (group A β-hemolytic streptococci), S. salivarius, S. sanguis, Staphylococcus aureus, S. epidermidis, S. haemolyticus, S. hominis, S. simulans, S. saprophyticus, methicillin/oxacillin-resistant (MRSA/ORSA) and methicillin/oxacillin-susceptible Staphylococci (MSSA/OSSA), Enterococcus (e.g., E. faecalis E. faecium, and E. hirae), vancomycin-resistant Enterococcus (VRE) and vancomycin-susceptible Enterococcus (VSE), Bacteroides fragilis, Propionibacterium acnes, Clostridium difficile (spore and vegetative cells), Selenomonas, Pseudomonas aeruginosa, Escherichia coli, Burkholderia cepacia, Proteus mirabilis, Gardnerella vaginalis, Klebsiella aerogenes, K. pneumoniae, K. pneumoniae multidrug resistant (MDR), Acinetobacter baumannii, A. baumannii MDR, Achromobacter xylosoxidans. Micrococus luteus, Ralstonia pickettii, Haemophilus influenza, and Serratia marcescens

Example fungi include, but are not limited to, Aspergillus niger, Candida albicans, C. aurus, C. dubliniensis, C. glabrata (formerly Torulopsis glabrata), C. guillermondii, C. kefyr (formerly C. pseudotropicalis), C. krusei, C. lusitaniae, C. tropicalis, Epidermophyton floccosum, Microsporum gypseum, M. canis, and Trichophyton mentagrophytes

Example viruses include, but are not limited to, those having a lipid component in their outer coat or have an outer envelope such as cytomegalovirus (CMV), human immunodeficiency virus (HIV), herpes simplex virus types 1 (HSV-1) and 2 (HSV-2), influenza virus, parainfluenza virus, variola virus (smallpox virus), vaccinia, norovirus, and coronavirus

According to some aspects, the certain time period may be a period of no more than about five minutes, optionally no more than about four minutes, optionally no more than about three minutes, optionally no more than about two minutes, and optionally no more than about one minute.

According to some aspects, the certain time period may be no more than about 120 seconds, optionally no more than about 105 seconds, optionally no more than about 90 second, optionally no more than about 75 seconds, optionally no more than about 60 seconds, optionally no more than about 45 seconds, optionally no more than about 30 seconds, and optionally no more than about 15 seconds.

It should be understood that “an acceptable log reduction” may be microbe-dependent. For example, an acceptable log reduction as described herein may refer to an acceptable log reduction of one type of microbe present on a surface (e.g., present in a body cavity or at an external wound site), a combination of two more types of microbes present on a surface, or total microbes present on a surface.

According to some aspects, an acceptable log reduction may be at least about 1.0, optionally at least about 1.1, optionally at least about 1.2, optionally at least about 1.3, optionally at least about 1.4, optionally at least about 1.5, optionally at least about 1.6, optionally at least about 1.7, optionally at least about 1.8, optionally at least about 1.9, optionally at least about 2.0, optionally at least about 2.1, optionally at least about 2.2, optionally at least about 2.3, optionally at least about 2.4, optionally at least about 2.5, optionally at least about 2.6, optionally at least about 2.7, optionally at least about 2.8, optionally at least about 2.9, optionally at least about 3.0, optionally at least about 3.1, optionally at least about 3.2, optionally at least about 3.3, optionally at least about 3.4, optionally at least about 3.5, optionally at least about 3.6, optionally at least about 3.7, optionally at least about 3.8, optionally at least about 3.9, optionally at least about 4.0, optionally at least about 4.1, optionally at least about 4.2, optionally at least about 4.3, optionally at least about 4.4, optionally at least about 4.5, optionally at least about 4.6, optionally at least about 4.7, optionally at least about 4.8, optionally at least about 4.9, and optionally at least about 5.0.

According to some aspects, the antiseptic agent may be present in the antiseptic solution in a concentration sufficient to provide an acceptable log reduction of microbes in a certain time period as described herein. According to some aspects, the antiseptic agent may be present in the antiseptic solution at a concentration of between about 0.001 and 5% w/v, optionally between about 0.001 and 2.5% w/v, optionally between about 0.001 and 1% w/v, optionally between about 0.001 and 0.1% w/v, optionally between about 0.001 and 0.01% w/v, optionally between about 0.01 and 5% w/v, optionally between about 0.01 and 2.5% w/v, optionally between about 0.01 and 2% w/v, optionally between about 0.01 and 1.5% w/v, optionally between about 0.01 and 1% w/v, and optionally about 0.5% w/v.

According to some aspects, the antiseptic agent may be present in the antiseptic solution at a concentration of between about 0.1 and 0.9% w/v, optionally between about 0.2 and 0.8% w/v, optionally between about 0.3 and 0.7% w/v, and optionally between about 0.4 and 0.6% w/v.

According to some aspects, the antiseptic agent may be present in the antiseptic solution at a concentration of between about 0.1 and 1% w/v, optionally between about 0.2 and 1% w/v, optionally between about 0.3 and 1% w/v, and optionally between about 0.4 and 1% w/v.

It should be understood that according to some aspects, the lavage fluid is not necessarily an antiseptic solution as described herein and may be any medically acceptable fluid configured to perform a lavage process as described herein. In one non-limiting example, the lavage fluid may comprise a saline solution. The saline solution may comprise water and sodium chloride in a medically acceptable concentration, such as between about 0.1 and 1%, w/v, optionally about 0.45% w/v, and optionally about 0.9% w/v.

According to some aspects, the lavage fluid, such as an antiseptic solution as described herein, may comprise a visualizing aid. As used herein, the term “visualizing aid” refers to a component in a lavage fluid configured to aid in visualizing the application of the lavage fluid. Example visualizing agents include, but are not limited to, tinting agents, staining agents, and radiopaque agents. It should be understood that the visualizing agent may be the same as or different from one of the other components of the lavage fluid. For example, the antiseptic agent may function as a visualizing agent. Additionally or alternatively, the lavage fluid may comprise a visualizing agent that is disparate from the antiseptic agent.

According to some aspects, the lavage fluid may comprise a tinting agent. As used herein, the term “tinting agent” refers to a component sufficient to provide an observable color to a fluid. The tinting agent may be sufficient to allow visualization of the lavage fluid upon application to a surface. In some non-limiting examples, the tinting agent may comprise an anionic tinting agent, such as an anionic dye. The anionic dye may be any dye suitable for medical use, such as dyes approved by the Food and Drug Administration for use in food, drugs, and/or cosmetics (i.e., “D&C” or “FD&C” dyes). Example anionic dyes include, but are not limited to, FD&C Blue No. 1 (Brilliant Blue FCF), FD&C Blue No. 2 (Indigo Carmine), FD&C Green No. 3 (Fast Green FCF), FD&C Red No. 3 (Erythrosine), FD&C Red No. 40 (Allura Red), FD&C Yellow No. 5 (Tartrazine), FD&C Yellow No. 6 (Sunset Yellow FCF), D&C Yellow No. 8 (Fluorescein), D&C Orange No. 4, and combinations thereof. Combinations may be implemented to arrive at a particular color. For example, an orange tint may comprise both FD&C Red No. 40 and D&C Yellow No. 8. Additionally or alternatively, the tinting agent may comprise a chemical compound that is observable upon exposure to visible and/or non-visible light, including, but not limited to, vitamin B-12, medical honey, fluorescent polymeric nanoparticles, water soluble luminescent carbon nanodots, quinine, and combinations thereof.

According to some aspects, the lavage fluid, such as an antiseptic solution as described herein, may comprise a staining agent. As used herein, the term “staining agent” refers to a component sufficient to temporarily or permanently color a surface with which it comes in contact.

According to some aspects, the lavage fluid, such as an antiseptic solution as described herein, may comprise a radiopaque agent. As used herein, the term “radiopaque agent” refers to a component that is opaque to the radio wave and x-ray portion of the electromagnetic spectrum sufficient for visualization. In some non-limiting examples, the radiopaque agent may comprise barium, iodine, iron oxide nanoparticles, gadolinium complex nanospheres, silica nanospheres, and combinations thereof.

According to some aspects, the lavage fluid, such as an antiseptic solution as described herein, may be basic, neutral, or acidic. According to some aspects, the lavage fluid may have a pH of between about 1 and 8, optionally between about 1 and 7, optionally between about 1 and 6, and optionally between about 2 and 5.5.

According to some aspects, the lavage fluid, such as an antiseptic solution as described herein, may comprise a buffer system. As used herein, the term “buffer system” refers to a component present in a composition or solution which may provide a resistance to significant change in pH caused by a strong acid or base. A buffer system may comprise a single agent or more than one agent, such as a weak acid and its conjugate base. A buffer system may provide a resistance to a significant pH change by interacting with a strong acid or strong base in a composition or solution, thereby at least partially preventing the pH of the composition or solution from changing significantly.

Generally, a buffer system has one or more buffer ranges wherein the buffer system has the ability to provide resistance to significant pH change. When a composition or solution comprising the buffer system has a pH inside the buffer system's buffer range, the pH of the composition or solution will not change significantly with the addition of equimolar amounts of a strong acid or strong base.

The buffer range of a buffer system is related to the acid dissociation constant (Kₐ) of one or more weak acids comprised by the buffer system. The term “acid dissociation constant” refers to the equilibrium constant of a dissociation reaction of an acid. The midpoint of a buffer range for a buffer system is generally about the logarithmic measure of the acid dissociation constant (i.e., the pKₐ, equal to −log₁₀ Kₐ) of a weak acid comprised by the buffer system.

According to some aspects, the lavage fluid, such as an antiseptic solution as described herein, may comprise a stabilizing agent. As used herein, the term “stabilizing agent” refers to any component that supports the stability of a lavage fluid not otherwise explicitly described herein.

The device according to the present disclosure comprises a body configured to contain a lavage fluid as described herein. According to some aspects, the body may be compressible. As used herein, the term “compressible” refers to the ability to reversibly reduce in volume without unacceptable changes, such as an unacceptable permanent change to size, to shape, and/or to one or more of the properties as described herein. According to some aspects, the body may be configured such that upon compression, at least a portion of the antiseptic agent contained therein is dispensed. It should be understood that as used herein, “dispense” (alternatively referred to as “discharge”) may refer to transferring the lavage fluid to an application member in fluid communication with the body and/or it may refer to transferring the lavage fluid from an application member to a surface.

According to some aspects, the body may be collapsible. As used herein, the term “collapsible” refers to the ability to permanently reduce in volume. For example, a collapsible body as described herein may have a first volume when a first volume of fluid is contained therein. When at least a portion of the fluid is dispensed, the collapsible body may collapse to have a second volume, the second volume being less than the first volume. It should be understood that a collapsible body will advantageously reduce the volume of waste (e.g., the volume of the body after the fluid therein has been dispensed). A collapsible body may further provide for a more efficient fluid discharge.

According to some aspects, the body may be configured to allow at least a 10% reduction in volume when compressed and/or collapsed, optionally at least a 20% reduction in volume, optionally at least a 30% reduction in volume, optionally at least a 40% reduction in volume, optionally at least a 50% reduction in volume, optionally at least a 60% reduction in volume, optionally at least a 70% reduction in volume, optionally at least a 80% reduction in volume, optionally at least a 90% reduction in volume, and optionally at least a 99% reduction in volume.

According to some aspects, the body may comprise a body material that is compatible with the lavage fluid contained therein, that is, a material that does not chemically or physically react with the lavage fluid or otherwise render the lavage fluid unfit for medical use.

According to some aspects, the body material may be sufficient to prevent unacceptable vapor or antiseptic loss from a lavage fluid contained therein over a certain period of shelf life. It should be understood that “unacceptable vapor or antiseptic loss” may be a loss that results in the lavage fluid becoming unsuitable for its intended use. Vapor or antiseptic loss may result from, for example, adsorption or absorption of the antiseptic by a material (e.g., by the body material), evaporation of solution, evaporation of a component of a solution (e.g., an antiseptic agent of an antiseptic solution), or a combination thereof. In one non-limiting example wherein the lavage fluid comprises water and iodine as described herein, the body material may be sufficient to prevent water vapor loss and/or iodine loss over a certain period of shelf life.

As used throughout this application, the term “shelf life” refers to the length of time that a product (e.g., an antiseptic solution) may be stored while remaining within the specifications required for the form, fit, and function of the product. Shelf life may be determined by measuring certain characteristics of the product that may indicate that the product is unfit for medical use. For example, shelf life may be determined by measuring the concentration of impurities in the product, the color change of the product, the concentration of insoluble particles in the product, the potency of an active agent contained by the product (e.g., an antiseptic agent), the concentration of one or more components of the product, the pH of the product, and/or the sterility of the product after storage in long-term storage conditions. As used herein, the term “long-term storage conditions” refers to environmental conditions sufficient for a product to be acceptably stored for more than 72 hours. According to some aspects, long-term storage conditions may refer to a temperature of about 25° C. and a relative humidity of about 60%. Additionally or alternatively, shelf life may be determined by measuring the concentration of impurities in the product, the color change of the product, the concentration of insoluble particles in the product, the potency of an active agent of the product, the concentration of one or more components of the product, the pH of the product, and/or the sterility of the product after storage at 37° C. and 65% relative humidity. Additionally or alternatively, shelf life may be determined by measuring the concentration of impurities in the product, the color change of the product, the concentration of insoluble particles in the product, the potency of an active agent of the product, the concentration of one or more components of the product, the pH of the product, and/or the sterility of the product after storage at between about 15 and 30° C., with excursions at a temperature of no more than about 40° C.

According to some aspects, the period of shelf life may be at least about 20 months, optionally at least about 21 months, optionally at least about 22 months, optionally at least about 23 months, optionally at least about 24 months, optionally at least about 25 months, optionally at least about 26 months, optionally at least about 27 months, optionally at least about 28 months, optionally at least about 29 months, optionally at least about 30 months, optionally at least about 31 months, optionally at least about 32 months, optionally at least about 33 months, optionally at least about 34 months, optionally at least about 35 months, optionally at least about 36 months, optionally at least about 37 months, optionally at least about 38 months, optionally at least about 39 months, and optionally at least about 40 months.

According to some aspects, the body material may be sufficient for sterilization by any known sterilization techniques useful according to the present disclosure, including moist heat sterilization (i.e., autoclaving), gas sterilization, gamma irradiation, electron-beam (e-beam) sterilization, aseptic manufacturing processes (e.g., aseptic filtration and/or blow-fill-seal operations), and combinations thereof. According to some aspects, a body material may be determined to be sufficient for sterilization if a container comprising the body material has a Sterility Assurance Level (SAL) of at least 10⁻⁶ after sterilization and provides an acceptable result upon integrity testing for the container closure after sterilization.

According to some aspects, the body material may have a sufficient mechanical strength such that the body provides an acceptable response to impact, vibration, shaking, or a combination thereof. According to some aspects, an acceptable response refers to a response compliant with ASTM D4169-16 (Standard Practice for Performance Testing of Shipping Containers and Systems), ASTM D4728-06 (Standard Test Method for Random Vibration Testing of Shipping Containers), ASTM D642-15 (Standard Test Method for Determining Compressive Resistance of Shipping Containers, Components, and Unit Loads), or any combination thereof. According to some aspects, the body material may be safe for biomedical use. For example, the body material may comply with ISO 10993 and/or with REACH requirements. According to some aspects, the body material may be sufficient to exhibit at least a portion of the characteristics described herein over a certain period of the lavage fluid's shelf life at a temperature of between about 15 and 30° C., with excursions at a temperature of no more than about 40° C. Additionally or alternatively, the body material may be sufficient to exhibit at least a portion of the characteristics described herein over a certain period of the lavage fluid's shelf life after storage at about 25° C. and 60% relative humidity. Additionally or alternatively, the body material may be sufficient to exhibit at least a portion of the characteristics described herein over a certain period of the lavage fluid's shelf life after storage at about 37° C. and 65% relative humidity.

The body material may be rigid or flexible. As used herein, the term “rigid” refers to a stiffness sufficient to resist deformation upon normal operating forces. As used herein, the term “flexible” refers to the ability to bend or compress under normal operating forces.

Example body materials include, but are not limited to, glass, plastic, paper, foil, and any combination thereof. Example plastics useful according to the present disclosure include, but are not limited to, high-density polyethylene (HDPE), low-density polyethylene (LDPE), polypropylene, polystyrene, nylon, and any combination thereof. According to some aspects, the body material may be a lined and/or coated material, such as a lined and/or coated paper.

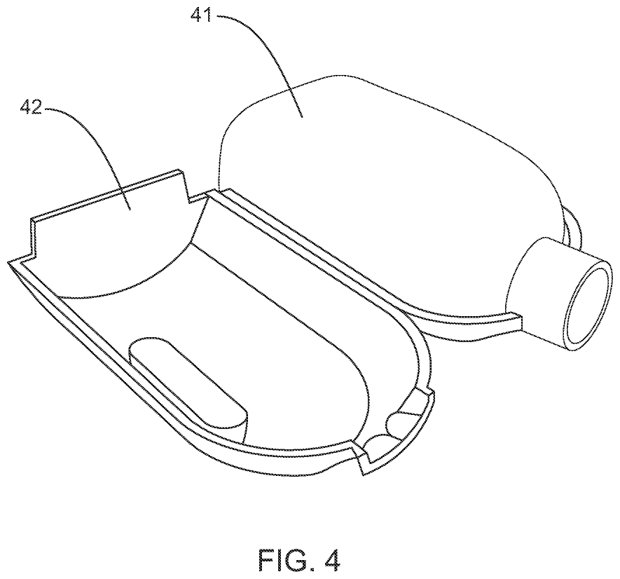

According to some aspects, the body may be provided with an outer casing. For example, FIG. 4 shows a body 41 comprising a flexible body material. Body 41 may be provided with an outer casing 42, which may be permanent or removable in relation to body 41. According to some aspects, outer casing 42 may be rigid, thus functioning to protect body 41 during storage and/or use. Outer casing 42 may additionally or alternatively function to distinguish body 41 from similar devices used in medical settings, such as intravenous (IV) fluid bags. In this way, outer casing 42 may reduce the risk of inadvertent misuse of body 41.

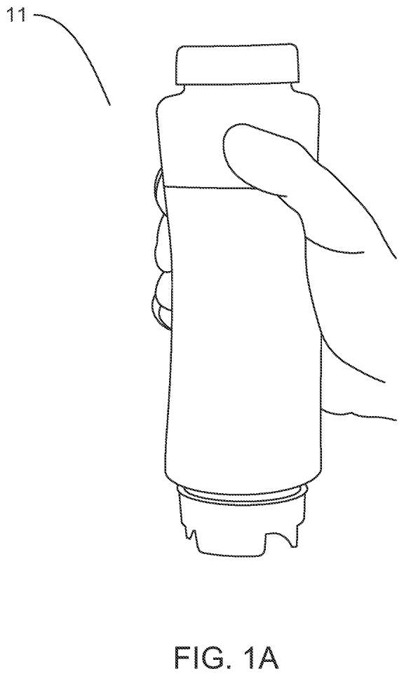

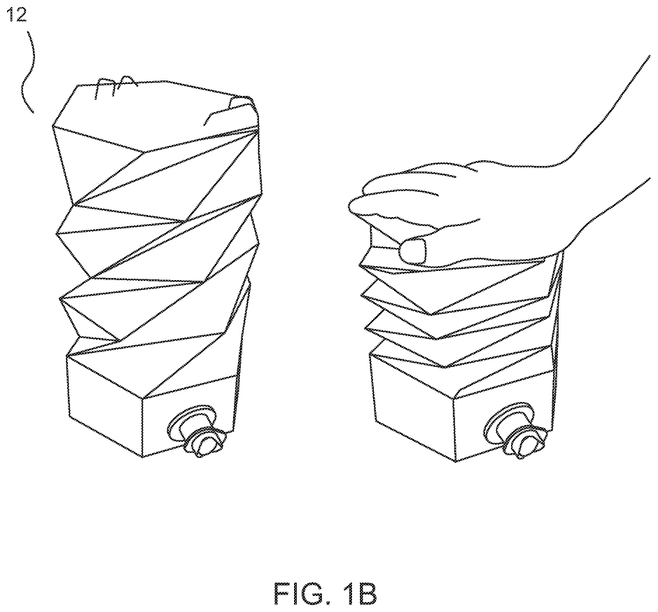

The body according to the present disclosure is configured to dispense a lavage fluid, such as an antiseptic solution, contained therein via one or more mechanisms. According to some aspects, the body may be configured to dispense the lavage fluid upon compression as described herein. For example, as shown in FIG. 1A, body 11 may be configured to dispense at least a portion of the lavage fluid contained therein in response to compression, such as squeezing. Additionally or alternatively, body 12 may be configured to dispense at least a portion of the lavage fluid contained therein in response to longitudinal compression, as shown in FIG. 1B.

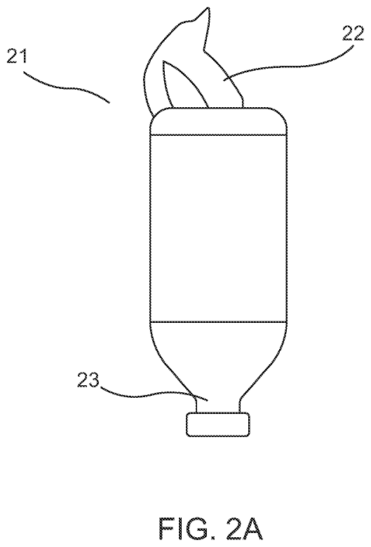

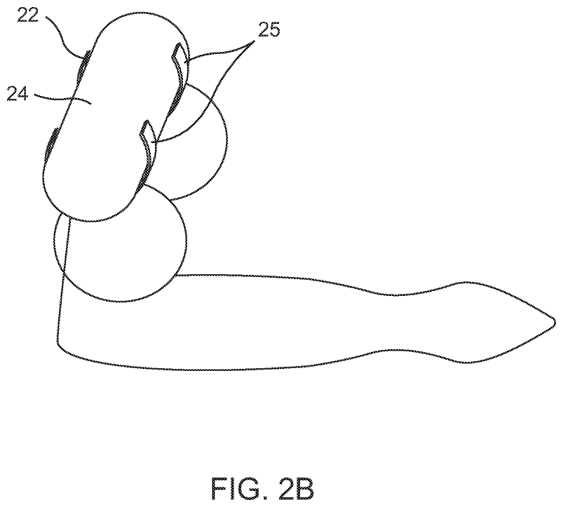

Additionally or alternatively, the body may be configured to dispense at least a portion of the lavage fluid contained therein upon orienting the body in a certain orientation. For example, as shown in FIG. 2A, body 21 may comprise an aperture 23 through which lavage fluid may be dispensed. In this example, body 21 may be configured such that when provided in a certain orientation (e.g., wherein aperture 23 is provided at or near the bottom of the body in relation to the ground), at least a portion of the lavage fluid is dispensed by the force of gravity.

In the example shown in FIG. 2A, body 21 may comprise a positioning component 22 that allows the body to be arranged in a certain orientation. The positioning component 22 may be any component configured to position and/or fix the body in a selected orientation, such as a hook, strap, snap, button, tie, or combination thereof. The positioning component 22 may be integral to the body and/or may be a separate component configured to interact with the body, such as a strap attachable to the body. The positioning component 22 may be configured to interact with a second positioning component, such as an extension arm configured to interact with a hook comprised by and/or attached to the body.

FIG. 2B shows another example of a system according to the present disclosure. In this example, body 24 is configured to interact with a separate positioning component 22, which may comprise, for example, snaps 25. In this way, body 24 may be positioned relative to and fixed to a user's arm, such as the arm of a medical practitioner performing lavage. In this example, lavage fluid may be dispensed by the force of gravity as described herein and/or by a dispensing aid as will be described herein.

According to some aspects, the body may be configured to communicate with a dispensing aid, wherein the dispensing aid is configured to provide a force sufficient to at least partially dispense the lavage fluid contained in the body. For example, the dispensing aid may comprise a pump configured to move the lavage fluid from the body. The pump may be a mechanical pump, a motorized pump, a vacuum pump, or any combination thereof.

It should be understood that the body may be configured to dispense the lavage fluid via one or a combination of the mechanisms as described herein. For example, the body may be configured to dispense the lavage fluid upon compression in conjunction with the force of gravity. Additionally or alternatively, the body may be configured to dispense the lavage fluid upon compression and/or by the force of gravity in conjunction with the force created by the pump (including, but not limited to, a vacuum force created by the pump). According to some aspects, the body may be configured to selectably dispense the lavage fluid via one or more of the mechanisms as described herein. In one non-limiting example, the body may be configured to dispense the lavage fluid upon compression both with and without the force of a pump. In this way, the user may select a desired delivery mechanism based on physical limitations (e.g., the physical capabilities of the user), a desired fluid flow force, a desired fluid flow rate, a desired fluid flow pattern (e.g., pulsed or constant), or a combination thereof.

According to some aspects, the body may be configured to dispense at least about 75% of the lavage fluid contained therein, optionally at least about 80%, optionally at least about 85%, optionally at least about 90%, optionally at least about 95%, and optionally about 100%. The body may be configured to continually dispense the lavage fluid and/or to intermittently dispense the lavage fluid. In one non-limiting example, the body may be configured to intermittently dispense the lavage fluid such that the lavage fluid is only dispensed upon compression of the body and/or upon actuation of a dispensing aid such as a pump.

The body may be configured to contain a volume of lavage fluid sufficient to perform at least a portion of a lavage process. According to some aspects, the body may be configured to contain between about 250 and 2000 mL of fluid, and optionally between about 500 and 1000 mL. According to some aspects, the body may be configured to contain about 500 mL of fluid. According to some aspects, the body may be configured to contain about 1 L of fluid.

The body according to the present disclosure may comprise a connection portion configured to selectively place the body in fluid communication with an applicator member. As used herein, the term “connection portion” refers to a portion of the body configured to provide a secure connection between the body and an application member such that fluid (e.g., an antiseptic solution) may be controllably dispensed from the body to the application member.

In one example, the connection portion is configured to fix the body and the application member such that a first aperture comprised by the body is aligned with a second aperture comprised by the application member sufficient to provide fluid communication between the body and application member. The connection portion may comprise any connection types known in the art useful according to the present disclosure.

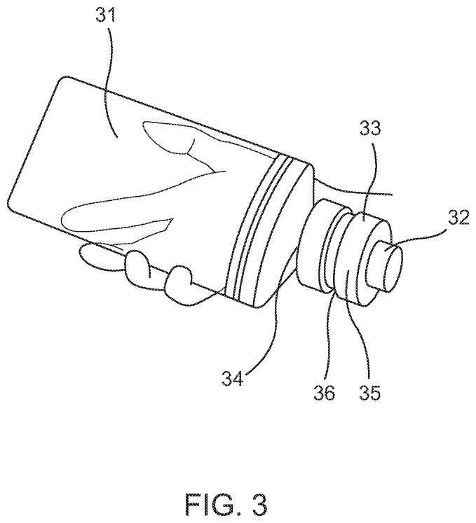

FIG. 3 shows an example of a connection portion 33 configured to connect a body 31 with an application member 32. In this example, connection portion 33 comprises protrusions configured to interact with corresponding protrusions comprised by the application member so as to form a screw connection, thereby allowing body 31 to be screwed to application member 32. It should be understood that in this example, screwing body 31 to application member 32 via connection portion 33 will align an aperture 34 of body 31 with an aperture 35 of application member 32 so as to provide fluid communication between body 31 and application member 32 when connected.

According to some aspects, the body may be provided with a removable lid, for example, a cap configured to interact with the connection portion of the body in place of the application member. It should be understood that the lid may prevent fluid discharge from the body, for example, during storage or transportation of the body.

According to some aspects, the connection portion may be provided with a fluid metering device, for example, a valve. The fluid metering device may be provided in communication with the body aperture (e.g., provided in the body aperture) sufficient to affect fluid flow from the body.

The present disclosure is also directed to a system comprising a body as described herein and one or more application members. The one or more application members may each be configured to apply a lavage fluid to a surface sufficient for a lavage process.

According to some aspects, the body may comprise a connection portion configured to interact with two or more different application members such that the system is adapted for interchanging application members. For illustrative purposes, taking the example shown in FIG. 3, the system may comprise a body 31 having a connection portion 33 as shown. The system may further comprise one or more application members each having a connection portion 36 with substantially the same size and shape such that each of the one or more application members may be interchangeably connected with body 31. In this way, a user may select from two or more application members based on lavage process preferences and requirements without requiring multiple body types. The system according to the present disclosure therefore beneficially allows a user to select from a variety of different application members, each of which may provide a unique fluid flow rate, fluid flow pattern, and/or fluid flow force, as will be described in more detail herein.

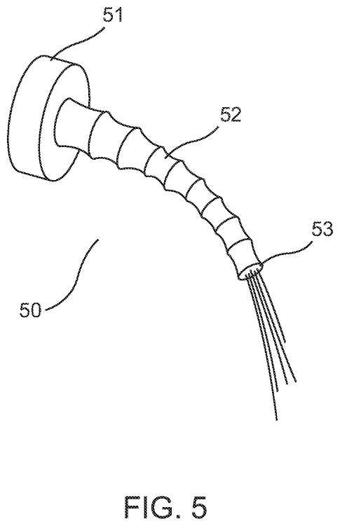

FIG. 5 shows one example application member 50 according to the present disclosure. As shown in FIG. 5, application member 50 may comprise a connection portion 51 and a discharge portion 52. Connection portion 51 may be configured to connect the application member 50 with a body as described herein. Discharge portion 52 may comprise one or more discharge apertures 53 configured to dispense a fluid (e.g., an antiseptic solution as described herein) onto a surface, such as a surgical site during a lavage process.

In the example shown in FIG. 5, discharge portion 52 may comprise a semi-flexible conduit such that the shape and/or orientation of the conduit is adjustable. In this way, the angle and/or direction of fluid discharge may be adjusted before and/or during a lavage process. As used herein, the term “semi-flexible” refers to the ability to bend or compress in addition to the ability to maintain shape when subjected to operating pressure, such as the pressure from fluid flow and/or the handling by a user. According to some aspects, the degree of flexibility of a semi-flexible component may depend at least in part on the application member material, the shape of the discharge portion, the length of the discharge portion, or a combination thereof. It should be understood that application member 50 as shown in FIG. 5 advantageously provides control of the flow path of a dispensed fluid such that a user may direct a fluid (e.g., an antiseptic solution) toward irregularly shaped and/or difficult to reach surfaces, such as irregularly shaped and/or difficult to reach surgical sites.

Example rigid application member materials useful according to the present disclosure include, but are not limited to, high-density polyethylene (HDPE), low-density polyethylene (LDPE), polyethylene, polypropylene, acrylonitrile butadiene styrene (ABS), polyurethane, polyether ether ketone (PEEK) and similar crystalline materials, thin walled metals, and combinations thereof. Example flexible application member materials useful according to the present disclosure include, but are not limited to, polyvinyl chloride (PVC), polypropylene, polyurethanes, silicones, and combinations thereof. Example semi-flexible application member materials useful according to the present disclosure include, but are not limited to, polyvinyl chloride (PVC), polyurethane, and combinations thereof. Additionally or alternatively, the application member material may comprise a laminate of one or more materials configured to provide a selected flexibility.

According to some aspects, the flexibility of one or more components of the application member (e.g., a discharge portion thereof) may depend at least in part on the application member material as described herein. Additionally or alternatively, the flexibility of one or more components of the application member may at depend at least in part on the presence of a structural element comprised by the application member, such as a metal wire embedded in the application member (e.g., embedded in a discharge portion thereof). Additionally or alternatively, the flexibility of the application member may depend at least in part on one or more other properties and/or dimensions of the application member. For example, the application member may comprise a conduit discharge portion as described herein, wherein the flexibility of the conduit discharge portion may depend at least in part on the thickness and/or hardness of the conduit wall(s). In one non-limiting example, a conduit having a durometer of about 100A may be rigid, a conduit having a durometer of about 80A may be semi-flexible, and a conduit having a durometer of about 45A may be flexible.

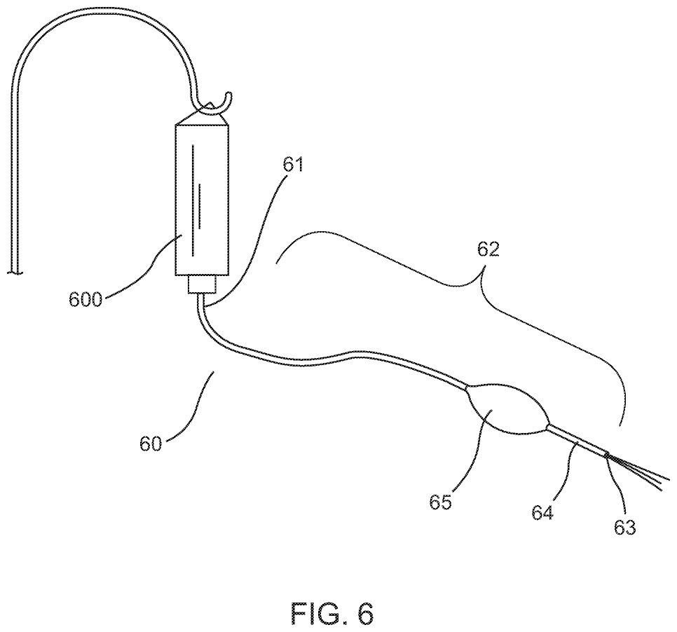

FIG. 6 shows another example application member 60 according to the present disclosure. As shown in FIG. 6, the application member 60 may comprise a connection portion 61 and a discharge portion 62. Connection portion 61 may be configured to connect application member 60 with body 600 as described herein. Discharge portion 62 may comprise one or more discharge apertures 63 configured to dispense a fluid (e.g., an antiseptic solution) onto a surface, such as a surgical site during a lavage process. It should be understood that discharge portion 62 may comprise a conduit 64 that may be a semi-flexible conduit as described in relation to FIG. 5, a flexible conduit, or a rigid conduit.

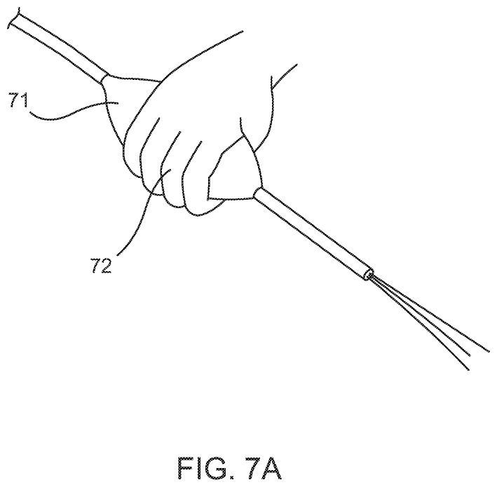

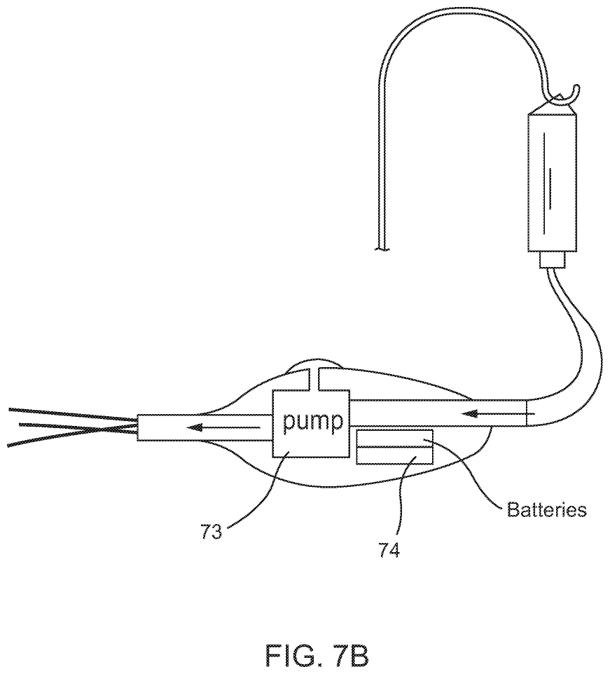

As shown in FIG. 6, application member 60 may further comprise a dispensing aid 65 as described herein, such as a pump. Dispensing aid may be a mechanical pump, for example, as shown in FIG. 7A. FIG. 7A shows a hand pump 71 that moves fluid upon compression by a user's hand 72. Additionally or alternatively, dispensing aid may be a motorized pump as shown in FIG. 7B. FIG. 7B shows a motorized pump 73 that moves fluid via electrical energy produced by, for example, batteries 74.

It should be understood that application member 60 having dispensing aid 65 as described herein may dispense a lavage fluid (e.g., an antiseptic solution) from body 600 upon actuation of dispensing aid 65 (e.g., actuation of a pump as described herein). Additionally or alternatively, dispensing aid 65 may function to dispense fluid from body 600 in conjunction with the force of gravity. For example, FIG. 6 shows an example body 600 similar to the body shown in FIG. 2A, that is, a body configured such that at least a portion of the lavage fluid contained therein is dispensed by the force of gravity when provided in a certain orientation. It should be understood that the dispensing aid will advantageously allow a user to control the fluid flow force, the fluid flow rate, and/or the fluid flow pattern (e.g., pulsed or constant) of the dispensed lavage fluid.

While the examples shown in FIGS. 5 and 6 show discharge portions having one discharge aperture, it should be understood that the discharge portion may comprise two, three, four, or more discharge apertures. Each of the discharge apertures may be the same size as or a different size from one or more of the other discharge apertures. Additionally or alternatively, each of the discharge apertures may have the same shape as or a different shape from one or more of the other discharge apertures. The shape and/or size of the one or more discharge apertures may be selected to provide a certain fluid flow force, fluid flow rate, and/or fluid flow pattern. According to some aspects, the shape and/or size of the one or more discharge apertures may be adjustable such that the fluid flow force, fluid flow rate, and/or fluid flow pattern of a dispensed fluid may be adjustable.

According to some aspects, the application member may comprise a multifunctional application member configured to provide more than one selectable functions.

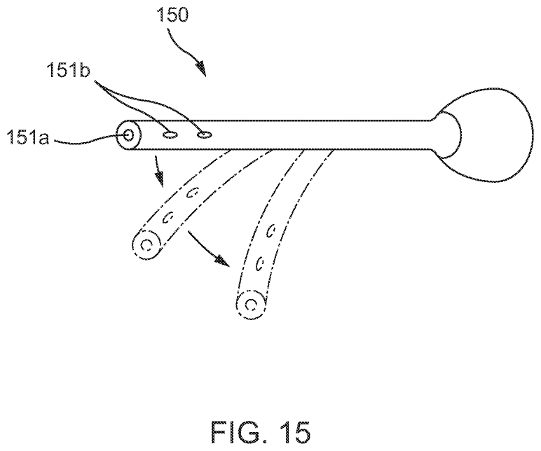

For example, according to some aspects, the application member may be configured to provide more than one selectable fluid flow pattern as described herein. In one non-limiting example, the application member may be configured to provide more than one fluid delivery angle. For example, as shown in FIG. 15, application member 150 may comprise one or more discharge apertures 151a, 151b as described herein. In this example, application member 150 may comprise a semi-flexible conduit as described herein (for example, with respect to FIG. 5), wherein the semi-flexible conduit is configured to be bent to a desired shape prior to irrigation, and the semi-flexible conduit maintains that desired shape after it is bent (i.e., after pressure on the semi-flexible conduit is released). In this way, a user may select the angle at which a lavage fluid is dispensed via discharge apertures 151a, 151b (and thus the surface to be irrigated). In addition, application member 150 may allow access to otherwise difficult to reach areas by selecting a certain shape of application member 150 specifically to reach that area.

As described herein, each of the one or more discharge apertures may have a selected size and/or shape in order to provide a certain fluid flow force, fluid flow rate, and/or fluid flow pattern. For example, in the example shown in FIG. 15, application member 150 may comprise one or more discharge apertures 151a proximal the end of the conduit and one or more discharge apertures 151b provided through a side wall of the conduit. The size of aperture(s) 151a, 151b may each be about the same as one or more of the other aperture(s) or different. For example, aperture(s) 151a may be smaller than aperture(s) 151b such that a pressure of lavage fluid dispensed via aperture(s) 151b (i.e., in a direction perpendicular to a length of the conduit) is less than the pressure of lavage fluid dispensed via aperture(s) 151a (i.e., in a direction parallel to a length of the conduit). Alternatively, aperture(s) 151a may be bigger than aperture(s) 151b such that a pressure of lavage fluid dispensed via aperture(s) 151b is greater than the pressure of lavage fluid dispensed via aperture(s) 151a.

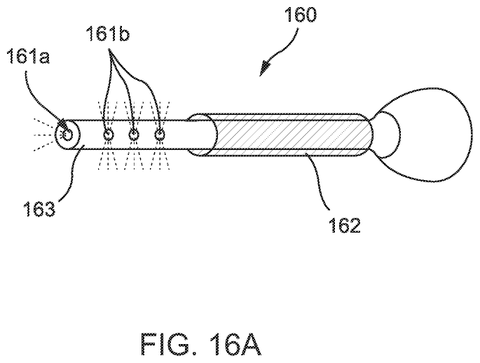

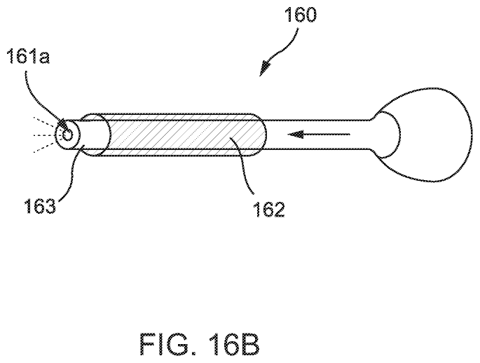

Additionally or alternatively, the multifunctional application member may comprise and/or may be configured to communicate with an obstruction component such that one or more, or each of the one or more, discharge apertures may be selectably obstructed in order to prevent or inhibit lavage fluid from passing therethrough. For example, FIG. 16A shows an application member 160 having one or more discharge apertures 161a, 161b, similar to the example shown in FIG. 15. Application member 160 may further comprise an obstruction component (e.g., slidable sheath 162) configured to fit snugly around conduit 163. While not shown, application member 160 may additionally or alternatively comprise an obstruction component comprising a slidable sheath configured to fit snugly within conduit 163. In this example, the slidable sheath may be moved along conduit 163 (for example, as shown in FIG. 16B) prior to and/or during a lavage process so as to selectively obstruct one or more of apertures 161b. One or more, or each of the one or more, apertures 161b may be completely obstructed such that lavage fluid is prevented from passing therethrough. In this way, lavage fluid may be selectably prevented from passing through one or more of apertures 161b, which may in turn increase the volume and/or pressure of lavage fluid dispensed through unobstructed apertures. Additionally or alternatively, one or more, or each of the one or more, apertures 161b may be partially obstructed such that lavage fluid is inhibited from passing therethrough. In this way, the pressure of lavage fluid dispensed through the unobstructed area of the partially obstructed aperture(s) may be increased, the direction of lavage fluid dispensed through the unobstructed area of the partially obstructed aperture(s) may be selected, and/or the volume and/or pressure of lavage fluid dispensed through unobstructed apertures may be increased.

While FIGS. 16A and 16B show an example obstruction component comprising a slidable sheath, the device and/or system according to the present disclosure may additionally or alternatively comprise a different obstructive component configured to selectively obstruct one or more discharge apertures. For example, the obstruction component may comprise a movable and reversibly fixed sheath (i.e., a movable sheath that may be reversibly fixed in place by a screw or other fixing mechanism) and/or one or more removable plugs and/or screws insertable in one or more discharge apertures. Additionally or alternatively, the application member may be provided with one or more pre-scored areas which may be removed prior to and/or during a lavage process in order to provide a selected number of discharge apertures. According to some aspects, each of the one or more pre-scored areas may comprise a biodegradable, non-toxic material, examples of which include, but are not limited to, polylactic acid (PLA).

It should be understood that while FIGS. 15, 16A, and 16B show a discharge aperture proximal an end of a conduit and one or more discharge apertures provided through a side of the conduit, the multifunctional application members according to the present disclosure are not necessarily limited to this arrangement. For example, the application member may comprise discharge aperture(s) only through a side wall of a conduit such that lavage fluid is dispensed only in a direction approximately perpendicular to a length of the conduit. Alternatively, the application member may comprise discharge aperture(s) only proximal an end of a conduit such that lavage fluid is dispensed only in a direction approximately parallel to a length of the conduit.

In addition, while FIGS. 15, 16A, and 16B show a conduit having a substantially cylindrical shape, the conduit may have a shape selected for a certain function. For example, the conduit may have any cross-sectional geometric shape (e.g., circular, rectangular, rectangular with rounded edges, oval) sufficient to perform a lavage process in a certain area of the body. Additionally or alternatively, the conduit may have a varying cross-sectional geometric shape along its length. For example, the conduit may have a first cross-sectional size or shape at a first end (for example, an end proximal a body as described herein) and a second cross-sectional size or shape at an opposite end. In one non-limiting example, the conduit may have a larger cross-sectional size at a first end and a smaller cross-sectional size at a second end, thereby allowing access to smaller areas to be irrigated during a lavage process.

In addition to or instead of the above functions, the application member may have a shape configured for acceptable obstruction maneuvering. As used herein, the phrase “obstruction maneuvering” refers to the maneuvering of any material that limits a practitioner's access to and/or view of an area to be irrigated during a lavage process. In some non-limiting examples, the obstructing material may be tissue within a surgical cavity. It should thus be understood that acceptable obstruction maneuvering requires the movement and/or removal of the obstructing material without unacceptable damage to the obstructing material and/or surrounding area.

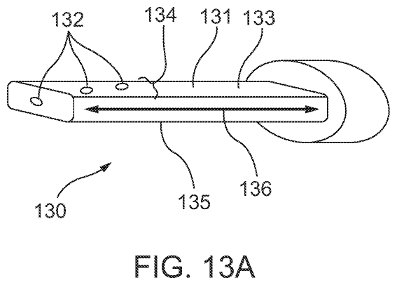

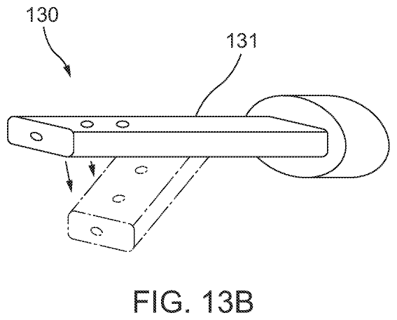

For example, FIG. 13A shows an example of a multifunctional application member 130 having a discharge portion 131 with one or more discharge apertures 132 as described herein. As shown in FIG. 13A, at least a portion of discharge portion 131 has a shape configured for acceptable obstruction maneuvering, and in particular, at least a first substantially smooth surface 133 and optionally at least a second substantially smooth surface 135 opposite the first substantially smooth surface 133. In this example, one or both of first substantially smooth surface 133 and second substantially smooth surface 135 may be substantially flat, as shown. It should be understood that while FIG. 13A shows first substantially smooth surface 133 and second substantially smooth surface 135 having a rectangular shape as shown, each surface may have any shape sufficient for acceptable obstruction maneuvering as described herein. For example, one or more dimensions of the first substantially smooth surface 133 and/or second substantially smooth surface 135, such as a width 134 or a length 136 thereof, may be selected based on the size and/or composition of the area to be irrigated during a lavage process.

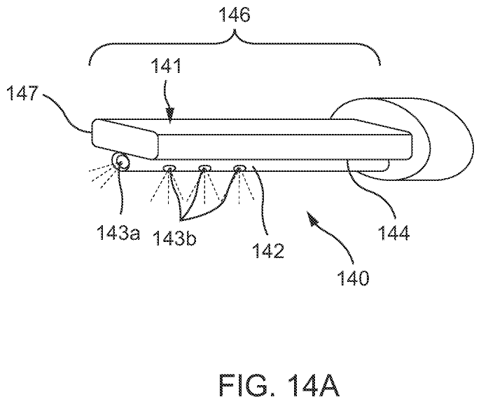

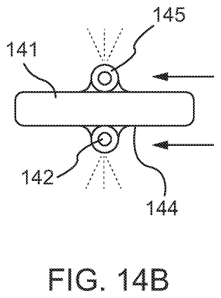

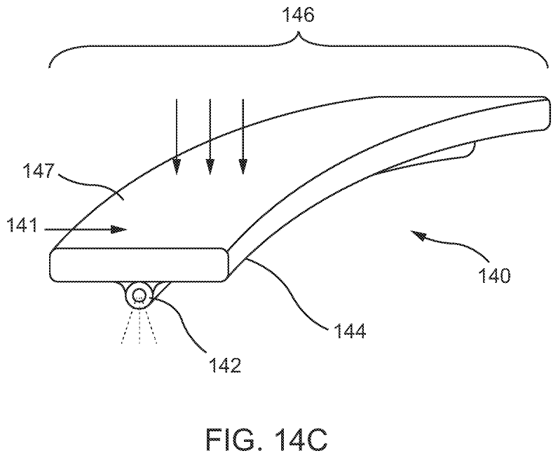

While FIG. 13A shows one or more discharge apertures 132 traversing first substantially smooth surface 133, one or more discharge apertures may additionally or alternatively be provided in a different position relative to the application member 130. For example, FIG. 14A shows an application member 140 similar to FIG. 13A having a discharge portion 146, discharge portion 146 comprising a maneuvering portion 147 having a first substantially smooth surface 141 and optionally a second substantially smooth surface 144 as described herein. In this example, second substantially smooth surface 144 may be provided with (e.g., attached to) a discharge conduit 142, such as a conduit as described in relation to FIGS. 5, 15, 16A, and/or 16B. In this example, discharge conduit 142 may comprise one or more discharge apertures provided at an end thereof 143a and/or one or more discharge apertures provided through a side thereof 143b. In another example, first substantially smooth surface 141 may be provided with a first discharge conduit 142 and second substantially smooth surface 144 may be provided with a second discharge conduit 145, as shown in FIG. 14B.

While FIGS. 13 and 14 show the first substantially smooth surface and the second substantially smooth surface as being substantially flat, the disclosure is not necessarily limited to this arrangement. For example, one or more of the substantially smooth surface(s) as described herein may be concave and/or convex, so long as the multifunctional application member is sufficient to acceptably maneuver obstructions as described herein. In this way, obstructions may be maneuvered without requiring an additional instrument and/or a practitioner's hand to be present in the body cavity during a lavage process. The efficiency of the lavage process may thus at least in part be increased as compared with application members without this feature.

According to some aspects, all or a portion of the multifunctional application member having a shape configured for acceptable obstruction maneuvering as described herein may be rigid, flexible, or semi-flexible as described herein. For example, FIG. 13B shows an application member 130 having a discharge portion 131 as described in relation to FIG. 13A. As shown in FIG. 13B, all or a portion of discharge portion 131 may be flexible or semi-flexible such that a user may select a shape of the application member prior to and/or during a lavage process based on the area to be irrigated, as described above. Similarly, as shown in FIG. 14C, application member 140 may comprise a discharge portion 146 comprising a maneuvering portion 147 having a first substantially smooth surface 141 and optionally a second substantially smooth surface 144. In this example, first substantially smooth surface 141 and/or second substantially smooth surface 144 may be provided with (e.g., attached to) a discharge conduit 142, as described herein. In this example, each of the maneuvering portion 147 and the discharge conduit 144 may be flexible and/or semi-flexible, as described in relation to FIG. 13B.

According to some aspects, the discharge portion as described herein may comprise a material suitable for use in the human body, and in particular, a low-friction material. In this way, the risk of inadvertent damage to an area to be irrigated by a lavage process may be reduced. Example low-friction materials useful according to the present disclosure include, but are not limited to, silicone, polyethylene, polypropylene, polytetrafluoroethylene (PTFE), and combinations thereof. According to some aspects, all or a portion of the discharge portion of a multifunctional application member may be formed from a low-friction material as described herein. Additionally or alternatively, all or a portion of the discharge portion may have a coating comprising a low-friction material as described herein. For example, all or a portion of the discharge portion may comprise any application member material as described herein (e.g., a metal) coated with a low-friction material, such as silicone, polytetrafluoroethylene (PTFE), and a combination thereof. Additionally or alternatively, the application member may comprise a coating having at least one material that is lubricious in the presence of fluids, including bodily fluids. Non-limiting examples of such lubricious materials include polyolefin heat shrink tubing and other heat shrinkable materials such as fluorinated ethylene propylene (FEP). According to some aspects, all or a portion of the discharge portion may be shaped to reduce the risk of inadvertent damage to an area to be irrigated by a lavage process. For example, all of a portion of the discharge portion may have smooth and/or rounded edges.

In addition to or instead of the above functions, the multifunctional application member may be configured to dispense lavage fluid as described herein and to also aid in material removal by the device or system, particularly the removal of material before and/or during a surgical procedure. Example materials to be removed by the device or system include, but are not limited to, microbes, debris (e.g., tissue, bone, bodily fluids, and extraneous material(s), such as particulate matter from a surgical operation), spent (i.e., previously dispensed) lavage fluid, and combinations thereof.

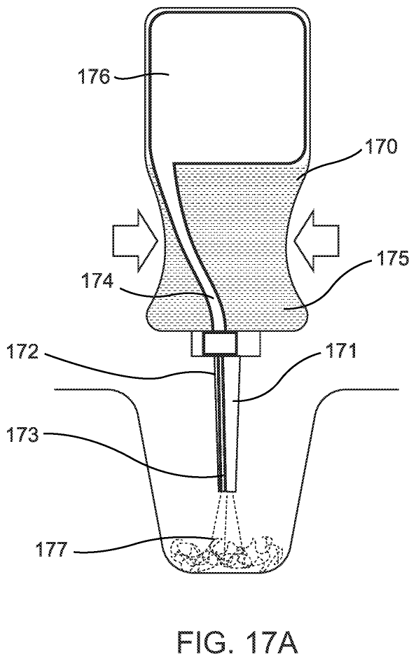

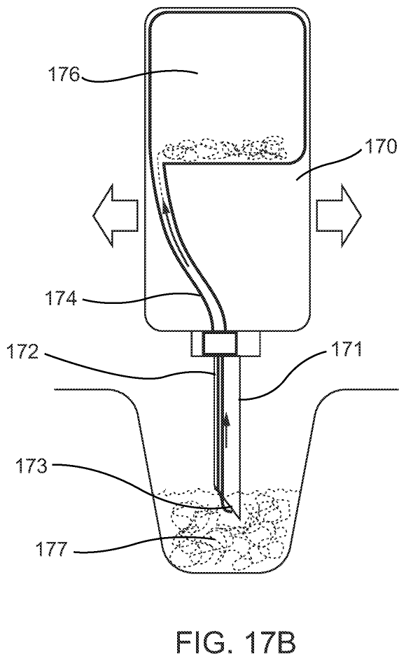

For example, the multifunctional application member may comprise one or more suction ports configured to intake material to be removed. In one non-limiting example, as shown in FIG. 17A, the application member 171 may comprise or may be configured to function in conjunction with a suction component 172, suction component 172 having at least one suction port 173 configured to intake material to be removed 177 (also referred to herein as waste). It should be understood that the at least one suction port 173 is separate from one or more discharge apertures comprised by application member 171 such that waste 177 does not contaminate a lavage fluid contained in the body. For example, in the example shown in FIG. 17A, the at least one suction port 173 may be in fluid communication with a fluid channel 174 that is separate from a fluid path along which lavage fluid 175 contained in a body 170 may be dispensed. As shown in FIG. 17A, fluid channel 174 may be in fluid communication with a waste receptacle 176 configured to house removed material. Waste receptacle 176 may be provided inside application member 171 and/or body 170 (as shown in FIG. 17A) or may be provided outside application member 171 and body 170. It should be understood that waste receptacle 176 and fluid channel 174 are each configured to keep removed material (i.e., waste 177) separate from lavage fluid 175 contained in body 170.

While FIG. 17A shows only one suction port 173, suction component 172 may have two, three, or more suction ports. Each of the more than one suction ports may be provided proximal at least one other suction port and/or may be provided at a different location along fluid channel from at least one other suction port.

According to some aspects, suction component 172 may be configured to intake waste via a suction force. The suction force may be provided, for example, by a vacuum provided in communication with the at least one suction port.

Additionally or alternatively, as shown in FIG. 17A, body 170 may be compressible as described herein, the compressible body configured such that, upon compression, at least a portion of the lavage fluid contained therein is dispensed. In this example, completely dispensing lavage fluid contained in the compressible body may require one or more re-equilibrium periods wherein gas from an external environment is pulled into the body sufficient to re-equilibrate the body's internal pressure prior to the next compression, as shown in FIG. 17B. Each of the one or more re-equilibrium periods in this example may provide a suction force to suction component 172 as described herein.

According to some aspects, each of the at least one suction ports as described herein may comprise one or more restrictive features, wherein the one or more restrictive features are configured to prevent unacceptable fluid passage through the suction port. For example, the suction port(s) may comprise a one-way valve having a first, closed position that prevents fluid passage therethough and a second, open position that allows fluid passage therethrough. In this example, the one-way valve may be provided in the first position when subjected to pressure from one direction (e.g., air pressure from inside the body upon compression of the body and/or pressure from waste contained in the suction component). The one-way valve may readily move to the second position when subjected to pressure from a different direction (i.e., air pressure from a surrounding environment, for example, during a re-equilibrium period as described herein, and/or waste from an area to be irrigated, such as a surgical site). In this way, the suction component may be configured to remove waste as described herein and may prevent waste to exit the suction component once removed, which may contaminate the area to be irrigated.

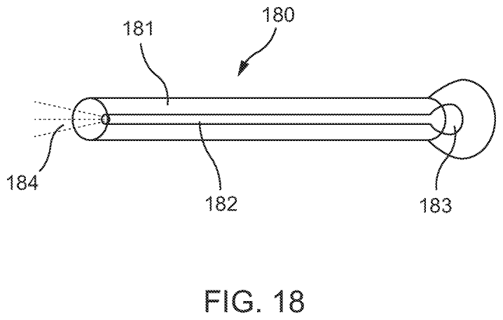

In addition to or instead of the above functions, the multifunctional application member may comprise or may be configured to communicate with a light component. For example, FIG. 18 shows a multifunctional application member 180 having a discharge portion 181 as described herein. Multifunctional application member 180 may further comprise or be configured to communicate with a light component 182. For example, in the example shown in FIG. 18, discharge portion 181 may be a conduit discharge portion as described herein, wherein light component 182 is provided within the conduit. However, it should be understood that the disclosure is not particularly limited to this arrangement. For example, all or a portion of light component 182 may be provided external to discharge portion 181.

Light component 182 may comprise a light source 183 configured to produce light 184 that is controllably emitted from light component 182. In this way, areas to be irrigated (e.g., surgical sites) may be more easily visualized without requiring separate light sources. In some non-limiting examples, light 184 provided by light component 182 may comprise visible light. For example, light source 183 may comprise one or more light-emitting diodes (LEDs). Additionally or alternatively, light 184 provided by light component 182 may comprise non-visible light, such as UV light.

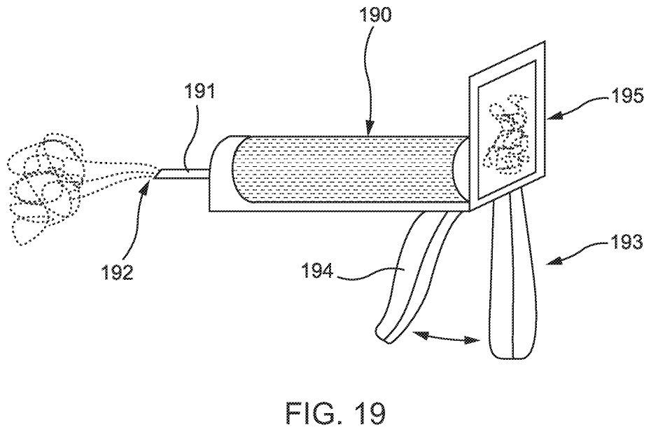

In addition to or instead of the above functions, the multifunctional application member may comprise or may be configured to communicate with a camera component, wherein the camera component is configured to provide a video image of an area to be irrigated. For example, FIG. 19 shows one non-limiting example wherein a body 190 is in fluid communication with a multifunctional application member 191 as described herein. Multifunctional application member 191 may comprise or be configured to communicate with a camera component 192 configured to capture a video image of an area to be irrigated. In this example, the captured video image may be provided on a separate display. For example, FIG. 19 shows an actuation device 193 configured to communicate with body 190 and multifunctional application member 192. Actuation device 193 may comprise an actuator 194 configured to dispense lavage fluid from body 190. Actuation device 193 may further comprise a display 195 configured to communicate with camera component 192 comprised by multifunctional application member 191 and/or comprised by actuation device 193 in order to display video images captured by camera component 192.

Additionally or alternatively, display 195 may be configured to provide one or more commands to the actuation device 193, multifunctional application member 191, and/or body 191 sufficient to control any of the functions described herein, for example, via a touchscreen. Example touchscreens useful according to the present disclosure include, but are not limited to, capacitive touchscreens, resistive touchscreens, and Surface Acoustic Wave (SAW) touchscreens. According to some aspects, the touchscreen may be in communication with a microprocessor such that information received by the touchscreen (e.g., a touch by a user's finger or other device) is communicated to the microprocessor. The microprocessor may be in communication with one or more solenoids and/or motor controls comprised by the actuation device such that, upon receiving a signal from the touchscreen as described herein, the microprocessor directs the one or more solenoids and/or motor controls to actuate and/or command one or more of the functions as described herein.

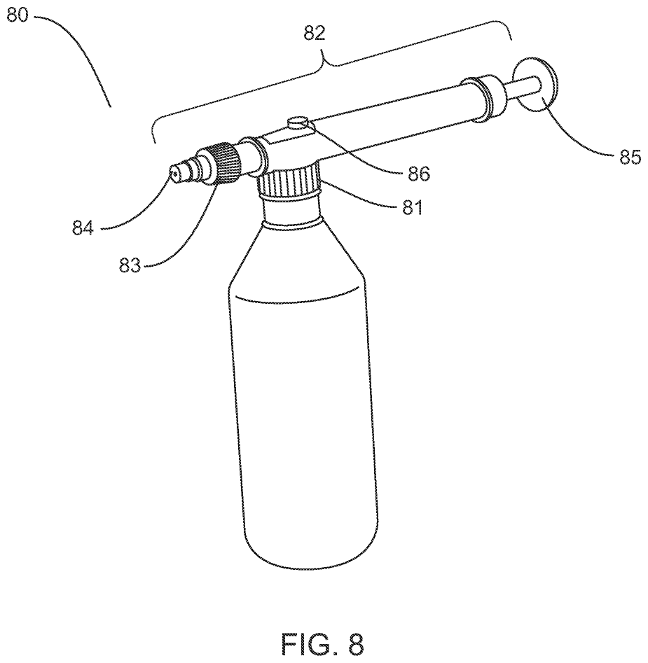

According to some aspects, the one or more discharge apertures may be provided in a nozzle portion of the discharge portion of an application member. For example, FIG. 8 shows a body 800 in fluid communication with an application member 80 having a connection portion 81 and a discharge portion 82 as described herein. As shown in FIG. 8, discharge portion 82 may comprise a nozzle 83 having one or more discharge apertures 84 as described herein. It should be understood that nozzle 83 may be removable and replaceable, thereby allowing the same application member 80 to interchangeably comprise at least two different nozzles 83. The system according to the present disclosure may thus comprise at least one application member and two or more interchangeable nozzles as described herein.

In the example shown in FIG. 8, application member 80 may further comprise a dispensing aid comprising a pump shaft 85 and an actuator 86, such as a button. In this example, nozzle 83 may be adapted to provide a mist of fluid in conjunction with pump shaft 85 upon actuation of actuator 86 by any mechanism known in the art. It should be understood that nozzle 83 may additionally or alternatively be configured to provide a stream of fluid, a spray of fluid, or a combination thereof.

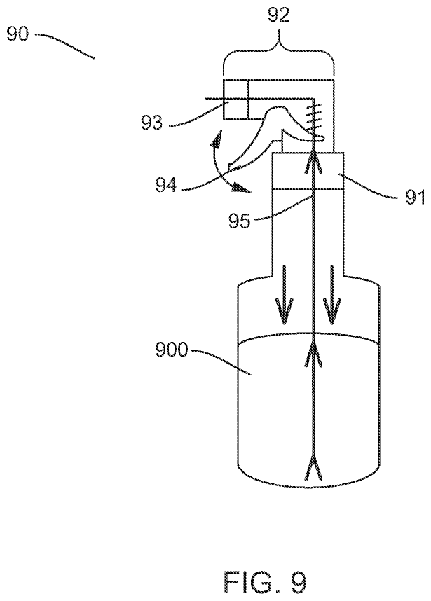

FIG. 9 shows another example of a system according to the present disclosure. As shown in FIG. 9, application member 90 may comprise a connection portion 91 and a discharge portion 92 as described herein. Discharge portion may comprise a nozzle 93 and an actuator 94, such as a trigger. In this example, application member 90 may further comprise a conduit 95 in fluid communication with a fluid contained in body 900 as described herein. In this example, body 900 may be pressurized. Upon actuation of actuator 94 (such as compressing the trigger), pressure in conduit 95 may drop below the pressure of body 900, thus forcing fluid from body 900 unto application member 90. Nozzle 93 may be configured to provide, for example, a fluid stream, a fluid mist, a fluid spray, or a combination thereof.

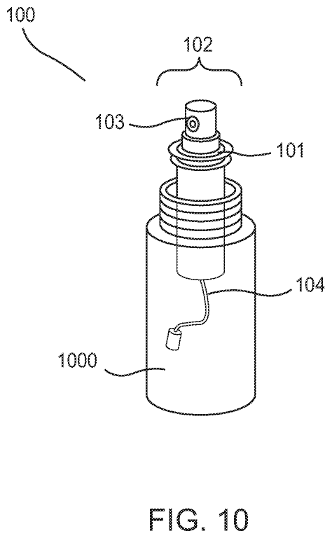

FIG. 10 shows another example of a system according to the present disclosure. As shown in FIG. 10, application member 100 may comprise a connection portion 101 and a discharge portion 102 as described herein. Discharge portion may comprise a nozzle 103. In this example, nozzle 103 may also function as an actuator, for example, by pressing nozzle 103 toward body 1000. Application member 100 may further comprise a conduit 104 in fluid communication with a fluid contained in body 1000. As described in relation to FIG. 9, body 1000 may be pressurized. Upon actuation of nozzle 103, pressure in conduit 104 may drop below the pressure of body 1000, thus forcing fluid from body 1000 unto application member 100, as described in relation to FIG. 9.

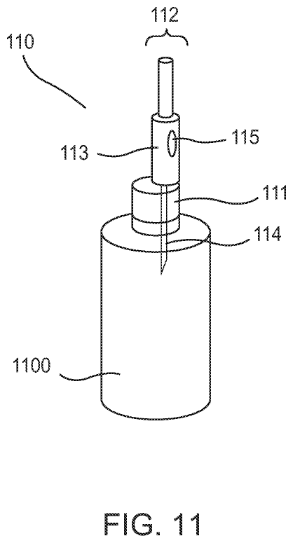

FIG. 11 shows another example system according to the present disclosure, including an application member 110, connection portion 111, discharge portion 112 including nozzle 113, and conduit 114, similar to the example shown in FIG. 10. FIG. 11 shows that nozzle 113 may further comprise an actuator 115, such as a button. As described in relation to FIGS. 9 and 10, body 1100 may be pressurized such that, upon actuation of actuator 115 (i.e., by pressing the button), pressure in conduit 114 may drop below the pressure of body 1100, thus forcing fluid from body 1100 into application member 110.

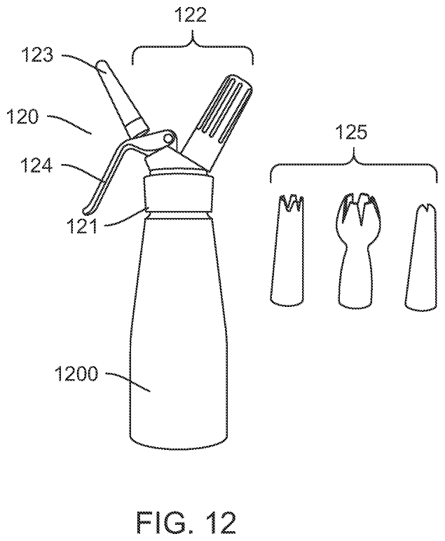

FIG. 12 shows another example of a system according to the present disclosure. As shown in FIG. 12, application member 120 may comprise a connection portion 121 and a discharge portion 122 as described herein. Discharge portion may comprise a first nozzle 123 and an actuator 124. As described in relation to FIGS. 9, 10, and 11, body 1200 may be pressurized. Additionally or alternatively, the system may comprise a cartridge containing a propellant (not shown) configured to provide an aerosol as known in the art. The propellant may be any propellant acceptable for medical use according to the present disclosure, including, but not limited to, carbon dioxide, nitrous oxide, nitrogen, helium, argon, air, and any combination thereof. As used herein, the term “air” refers to the natural atmosphere of the Earth.

The example system shown in FIG. 12 also shows a plurality of interchangeable nozzles 125 as described herein. It should be understood that each of the plurality of interchangeable nozzles 125 is configured to be interchangeable with nozzle 123, thus providing a single application member 120 and body 1200 configured to dispense a fluid to a surface via a variety of nozzles so as to provide a variety of different, selectable fluid flow rates, fluid flow patterns, and/or fluid flow forces, as described herein. It should be understood that the systems described herein may comprise at least a body configured to be in fluid communication with one or more different application members, each of the one or more different applications members having at least one discharge aperture, wherein the at least one discharge aperture is optionally comprised by a removable and replaceable nozzle, as described herein. It should be understood that the systems as described herein may thus be configured to deliver a lavage fluid to a surface via one or more different, selectable fluid flow rates, fluid flow patterns, and/or fluid flow forces, as described herein.

For example, the system may comprise at least two different application members and/or at least two different nozzles as described herein, wherein each of the at least two different application members and/or at least two different nozzles are configured to provide a unique fluid flow rate, fluid flow pattern, and/or fluid flow force. According to some aspects, a single application member and/or a single nozzle may be configured to provide at least two unique fluid flow rates, fluid flow patterns, and/or fluid flow forces, such as by providing one or more discharge apertures with adjustable shapes and/or sizes, as described herein.

According to some aspects, the system may be configured to provide an acceptable fluid flow rate for a lavage process. As used herein, the term “fluid flow rate” refers to the rate at which a fluid is applied to a surface, such as to a human subject during a lavage process. The fluid flow rate may depend at least partially on the delivery mechanism (e.g., compressing the body, orientating the body, utilizing a dispensing aid, or a combination thereof, as described herein) and/or the properties of the application member and/or nozzle as described herein. According to some aspects, a fluid flow rate may be related to a fluid flow force. For example, an increased fluid flow rate may correspond with an increased fluid flow force, and vice versa. The system according to the present disclosure may be configured to provide at least two different, selectable fluid flow rates, optionally at least three, optionally at least four, and optionally at least five.

According to some aspects, the system may be configured to provide an acceptable fluid flow force for a lavage process. As used herein, the term “fluid flow force” refers to the force of a fluid acting on a surface, such as on a human subject during a lavage process. An acceptable fluid flow force may be determined based on the lavage process requirements. Example fluid flow forces useful according to the present disclosure include, but are not limited to, between about 10 and 50 g, and optionally between about 15 and 45 g. According to some aspects, the fluid flow force may be about 15 g. According to some aspects, the fluid flow force may be between about 30 and 45 g. Other example fluid flow forces useful according to the present disclosure include, but are not limited to, between about 1 and 15 psi (referred to herein as “low pressure”) and between about 35 and 70 psi (referred to herein as “high pressure”).

It should be understood that the fluid flow force provided by the systems as described herein may depend at least partially on the delivery mechanism and/or the properties of the application member and/or nozzle as described herein. The system according to the present disclosure may be configured to provide at least two different, selectable fluid flow forces, optionally at least three, optionally at least four, and optionally at least five. It should be understood that each of the selectable fluid flow forces may correspond with, for example, a specific delivery mechanism, a specific application member, a specific nozzle, or a combination thereof, as described herein. For example, one or more selectable flow forces may correspond with an application member having an actuator as described herein, such as a trigger, wherein each of the one or more selectable flow forces may correspond with a degree of trigger compression. In another example, one or more selectable flow forces may correspond with a nozzle having one or more discharge apertures, wherein each of the one or more selectable flow forces may correspond with the shape and/or size of the one or more discharge apertures.

According to some aspects, the system is configured to provide an acceptable fluid flow pattern for a lavage process. As used herein, the term “fluid flow pattern” refers to the pattern with which a fluid is dispensed from a device and/or applied to a surface, such as to a human subject during a lavage process. In some non-limiting examples, the fluid flow pattern may comprise a fluid mist (i.e., a suspension of finely divided fluid in a gas), a fluid stream (i.e., a steady succession of fluid), a fluid spray (i.e., finely divided fluid), or a combination thereof. The fluid flow pattern may be constant (e.g., fluid continually dispensed from a device and/or applied to a surface) or pulsed (e.g., the fluid intermittently dispensed from a device and/or applied to a surface).

A fluid flow pattern may additionally or alternatively refer to the angle at which a fluid flow path is dispensed from a device and/or applied to a surface. For example, a fluid flow path may have a fluid flow pattern that is about perpendicular to a longitudinal axis of a body as described herein.

Additionally or alternatively, a fluid flow pattern may refer to the geometric shape of a fluid path. It should be understood that the geometric shape of a fluid path refers to a shape defined by the cross-sectional view of a fluid flow path in any of the x-direction, y-direction, and z-direction.

It should be understood that the fluid flow pattern may depend at least in part on the delivery mechanism and/or the application member and/or nozzle as described herein. The system according to the present disclosure may be configured to provide at least two different, selectable fluid flow patterns, optionally at least three, optionally at least four, and optionally at least five. For example, one or more selectable flow patterns may correspond with a dispensing aid such as a pump, wherein the pump may be configured to provide a constant flow of fluid from the body and/or to provide a pulsed flow of fluid from the body. In another example, one or more selectable flow patterns may correspond with the application member's discharge portion, such as a discharge portion comprising a semi-flexible conduit as described herein. In this example, the one or more selectable flow patterns may comprise one or more fluid delivery angles corresponding with the shape and/or orientation of the semi-flexible conduit as described herein.

According to some aspects, one or more components of the system described herein may be provided in sterile packaging. As used herein, the term “sterile packaging” refers to packaging that provides a sterile environment so as to maintain sterility of a contained sterile product. Example sterile packaging includes, but is not limited to, sterile blister packaging; sterile safe-edge trays, sterile surgical trays, sterile customized thermoforms, and combinations thereof. It should be understood that one or more components of the system may be provided in the same sterile packaging and/or separate sterile packaging from at least one other component of the system. For example, a first component of the system may be contained in a first sterile packaging and a second component of the system may be contained in a second sterile packaging. In one non-limiting example, the system may comprise a body contained in a first sterile packaging and an application member contained in a second sterile packaging. It should be understood that providing one or more components of the system in different sterile packaging allows for the removal of each component of the system immediately prior to its use, thus preventing one or more components from prolonged exposure to an unsterile environment. In this way, a fully assembled sterile presentation of the system may be achieved.

The present disclosure is also directed to methods of using the devices and systems described herein. For example, the method may comprise providing a body containing a lavage fluid, wherein the body comprises a connection portion. The method may comprise placing the body in fluid communication with an application member, such as a multifunctional application member as described herein, and dispensing the lavage fluid as described herein sufficient to perform a lavage process. The method may further comprise removing waste from an area to be irrigated by the lavage process prior to or during the lavage process using the multifunctional application member.

While the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the disclosure. Therefore, the disclosure is intended to embrace all known or later-developed alternatives, modifications, variations, improvements, and/or substantial equivalents.

Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

Further, the word “example” is used herein to mean “serving as an example, instance, or illustration.” Any aspect described herein as “example” is not necessarily to be construed as preferred or advantageous over other aspects. Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. Nothing disclosed herein is intended to be dedicated to the public regardless of whether such disclosure is explicitly recited in the claims.

The word “about” is used herein to mean within ±5% of the stated value, optionally within ±4%, optionally within ±3%, optionally within ±2%, optionally within ±1%, optionally within ±0.5%, optionally within ±0.1%, and optionally within ±0.01%.

What is claimed is:

1. A system for applying a lavage fluid to a surface, the system comprising:
a body configured to house a lavage fluid; and
a multifunctional application member in fluid communication with the body,
wherein the multifunctional application member is configured to dispense the lavage fluid, and
wherein the multifunctional application member comprises a suction component having at least one suction port configured to intake material.

2. The system according to claim 1, wherein the suction component comprises a fluid channel in fluid communication with the suction port, and wherein the fluid channel is in fluid communication with a waste receptacle configured to house the material.

3. The system according to claim 2, wherein the fluid channel is separate from a fluid path along which the lavage fluid is dispensed.

4. The system according to claim 2, where the waste receptacle is provided inside the body.

5. The system according to claim 1, wherein the at least one suction port comprises a one-wave valve.

6. The system according to claim 1, wherein the body is compressible.

7. The system according to claim 1, wherein the material is selected from the group consisting of microbes, debris, spent lavage fluid, and combinations thereof.

8. A system for applying a lavage fluid to a surface, the system comprising:
a body configured to house a lavage fluid; and
a multifunctional application member in fluid communication with the body,
wherein the multifunctional application member comprises a discharge portion having one or more discharge apertures configured to dispense the lavage fluid, and
wherein the discharge portion comprises a maneuvering portion comprising at least one substantially smooth surface.

9. The system according to claim 8, wherein at least a portion of the one or more discharge apertures traverses the at least one substantially smooth surface.

10. The system according to claim 8, wherein at least a portion of the one or more discharge apertures is comprised by a discharge conduit, and wherein the discharge conduit is attached to the at least one substantially smooth surface.

11. The system according to claim 10, wherein the portion of the one or more discharge apertures are provided proximal an end of the discharge conduit and/or through a side wall of the discharge conduit.

12. The system according to claim 8, wherein the discharge portion comprises a semi-flexible application member material.

13. The system according to claim 8, wherein the discharge portion comprises a low-friction material.

14. A system for applying a lavage fluid to a surface, the system comprising:
a body configured to house a lavage fluid; and
a multifunctional application member in fluid communication with the body,
wherein the multifunctional application member comprises a conduit discharge portion having one or more discharge apertures configured to dispense the lavage fluid,
wherein at least a first portion of the one or more discharge apertures is provided through a side wall of the conduit discharge portion, and
wherein the multifunctional application member comprises an obstruction component configured to selectably obstruct each of the first portion of the one or more discharge apertures.

15. The system according to claim 14, wherein the obstruction component comprises a slidable sheath.

16. The system according to claim 14, wherein a second portion of the one or more discharge apertures is provided proximal an end of the conduit discharge portion.

17. The system according to claim 14, wherein the conduit discharge portion comprises a semi-flexible application member material.

18. A system for applying a lavage fluid to a surface, the system comprising:
a body configured to house a lavage fluid; and
a multifunctional application member in fluid communication with the body,
wherein the multifunctional application member comprises a discharge portion having one or more discharge apertures configured to dispense the lavage fluid, and
wherein the multifunctional application member comprises a light component configured to controllably emit a light provided by a light source.

19. The system according to claim 18, wherein the light comprises visible light.

20. The system according to claim 18, wherein the light comprises non-visible light.
